(19) United States
(12) Patent Application Publication
(10) Pub. No.: US 2014/0180405 A1
(43) Pub. Date: Jun. 26, 2014

(54) CURVATURE CHANGING ACCOMMODATIVE INTRAOCULAR LENS

(71) Applicant: NOVARTIS AG, BASEL (CH)

(72) Inventors: JOSEPH I. WEINSCHENK, III, FORT WORTH, TX (US); LAUREN DeVITA GERARDI, CHICAGO, IL (US); HARI SUBRAMANIAM, IRVINE, CA (US)

(73) Assignee: NOVARTIS AG, BASEL (CH)

(21) Appl. No.: 14/096,104

(22) Filed: Dec. 4, 2013

Related U.S. Application Data
(60) Provisional application No. 61/745,130, filed on Dec. 21, 2012.

(51) Int. Cl. A61F 2/16

(52) U.S. Cl. CPC A61F 2/1635
USPC 623/6.13

(57) ABSTRACT

A curvature changing accommodative intraocular lens is provided in which the anterior surface of the intraocular lens undergoes dynamic change in curvature to focus light from distant objects to those nearby. The lens utilizes fluid movement from bladders defined as junctions between haptic elements and lens element or bladders positioned between the haptic elements and lens element periphery to change the curvature.

This application claims the priority of U.S. Provisional Patent Application No. 61/745130 filed on Dec. 21, 2012.

### FIELD OF THE INVENTION

The invention relates generally to the field of intraocular lens (IOL), and more particularly, to accommodative IOLs.

### BACKGROUND OF THE INVENTION

Intraocular lenses (IOL) have been developed for implantation in a person's eye to replace the natural crystalline lens which/that has been clouded by cataract, for example. Current IOLs generally have been primarily monofocal i.e., they focus light from distant objects onto the retina to improve distance vision. To see near objects, however, such as a computer screen or print in a book, an individual with implanted monofocal IOLs often still has to use reading glasses.

Existing designs for IOLs simultaneously focus light from distant and near objects on to the retina. The individual's brain then determines whether it wants to see a near or distant object. One drawback of these IOLs is that the overall image contrast generally is reduced because less than 100% of the light reaching the retina is from either the near or the distant object.

Some presbyopic IOL designs are dynamic and undergo graded movement under the forces available from the accommodative mechanism of the eye. These IOLs comprise a dual lens system wherein at least one of the lenses moves longitudinally under accommodative stress so that nearer objects come into focus. A drawback of these IOLs is that they often do not offer full accommodation (defined as a minimum of 2.5D (Diopter)). In other words, they do not offer sufficient lens movement so that the focus from a distance object can be moved to an object about 40 cm from an individual's head (where 40 cm is an average distance desired for reading). Current IOL designs that incorporate longitudinal movement of the lens provide less than 1D of accommodation.

Accordingly, there is a need for dynamically accommodating intraocular lens that offers a full range of vision (infinity to about 40 cm) to the individual in which it is implanted.

### SUMMARY OF THE INVENTION

The present invention generally relates to an intraocular lens that is adapted to be inserted into a wearer's eye for adjusting the vision thereof. The intraocular lens may include a lens element comprising a lens body defining a chamber, and an optic membrane extending across the chamber. The intraocular lens may comprise at least one bladder in fluid communication with the chamber. The at least one bladder and/or the chamber of the lens body may contain a fluid material therewithin. The intraocular lens further will comprise at least one haptic element connected to or adapted to engage the at least one bladder. Movement of the at least one haptic element thus generally will cause movement of fluid between and/or within the at least one bladder and the chamber so as to vary a lens radius of the optic membrane. The variation of the lens radius of the optic membrane will cause focus of the lens element to be adjusted for distance vision or nearer objects. Such variation of the lens radius of the optic membrane can allow for full accommodation (>=2.5D) from distant objects to those near (40 cm or even closer) the eye; thereby exceeding the performance of current IOL designs (<=1D) that rely on longitudinal movement of one or two lenses.

Other objects and advantages of the invention will be apparent to those skilled in the art based on the following drawings and detailed description.

### BRIEF DESCRIPTION OF THE DRAWINGS

FIG. 1 is a perspective view of intraocular lens 100A, according to one embodiment of the present invention.

FIG. 1B is a bottom view of intraocular lens 100A.

FIG. 1C is a side view of intraocular lens 100A.

FIG. 2 is a schematic view of intraocular lens 100B implanted within the eye of a wearer.

FIG. 3A is a top view of intraocular lens 100B, according to an alternative embodiment of the present invention.

FIG. 3B is a bottom view of intraocular lens 100B.

Those skilled in the art will appreciate and understand that, according to common practice, the various features of the drawings discussed below are not necessarily drawn to scale, and that dimensions of various features and elements of the drawings may be expanded or reduced to more clearly illustrate the embodiments of the present invention described herein.

### DETAILED DESCRIPTION

As illustrated in the drawings, the intraocular lens (IOL) 100A, 100B formed according to the principles of the present invention, is designed to dynamically change the curvature of the lens implanted into the eye of a patient to focus light from distant objects to those nearby by responding to the natural accommodative forces of the eye. Accommodation is the process by which the eye changes optical power (by changing natural lens shape) to maintain focus on an object as its distance changes.

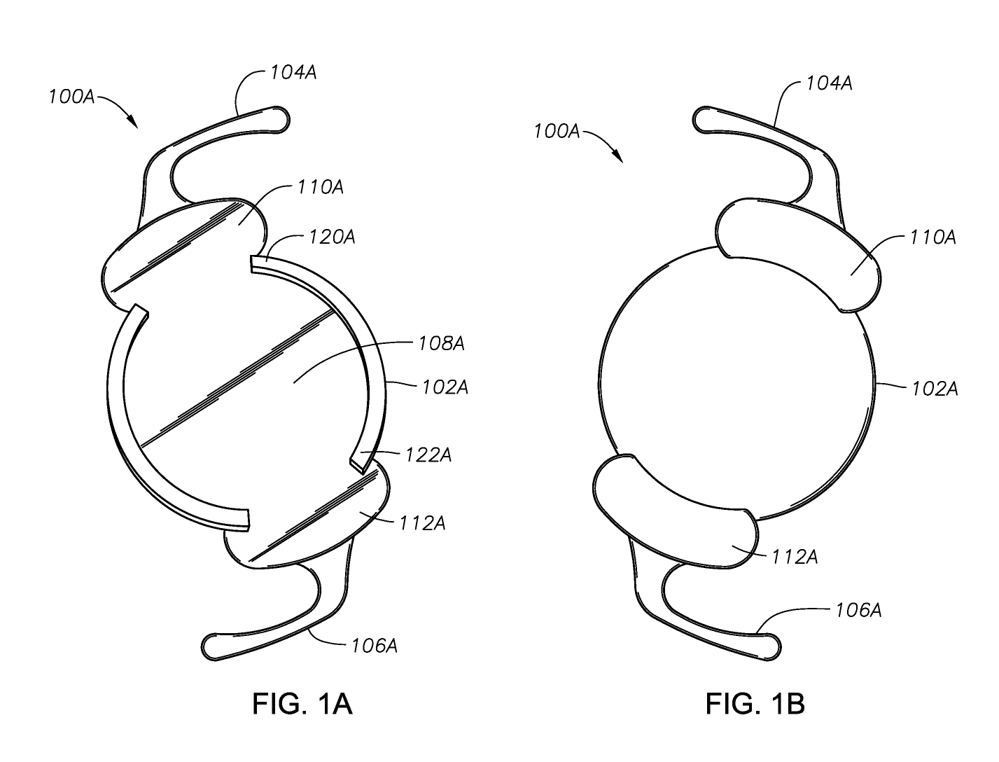

As illustrated in FIGS. 1A-3B, the IOL 100A/100B comprises a lens element 102A/102B, a first haptic element 104A/104B, and a second haptic element 106A/106B. Lens element 102A/102B generally is formed with a substantially hemispherically shaped body, which includes an internal chamber 108A/108B defined by an optic membrane (shown as 310A in the side view of FIG. 1C) extending over/across the chamber 108A/108B. Optic membrane 310A may be a soft membrane generally located on the anterior side of chamber 108A/108B.

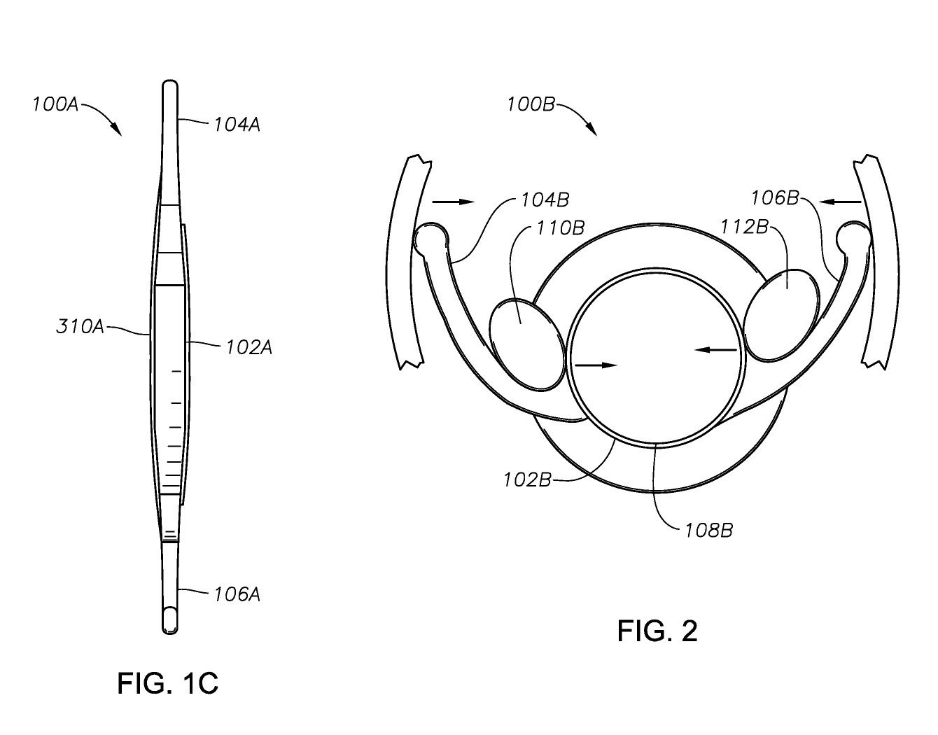

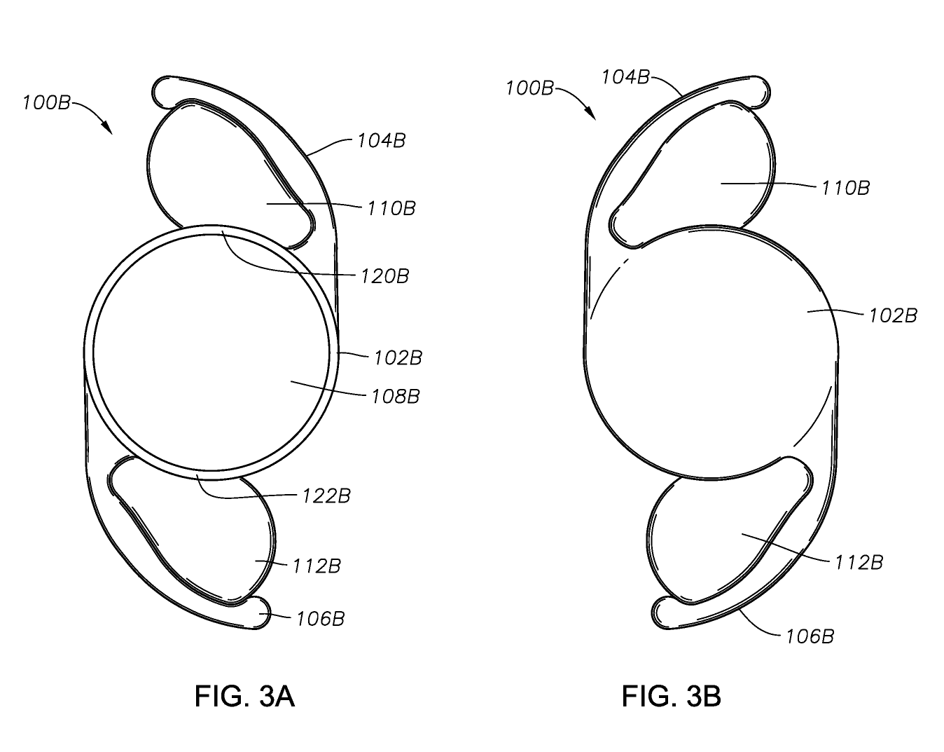

As indicated in FIGS. 2A and 3A, first haptic element 104A/104B and second haptic element 106A/106B may be connected to lens element 102A/102B on opposite sides thereof. In an embodiment as illustrated in FIG. 2A, for example, first haptic element 104A and second haptic element 106A may be formed integrally with lens element 102A. First haptic element 104A, second haptic element 106A, and lens element 102A may be molded in one-piece. In an alternative embodiment as illustrated in FIG. 3A, for example, first haptic element 104B, second haptic element 106B, and lens element 102B may be formed as separate pieces or components that are attached together by plasma bonding, adhesives, and/or other bonding techniques. When implanted in the eye, first haptic element 104A/104B and second haptic element 106A/106B support lens element 102A/102B within the capsular bag.

IOL 100A/100B further will include a first bladder 110A/110B, and typically a second bladder 112A/112B as indicated in FIGS. 2A and 3A. First bladder 110A/110B and second bladder 112A/112B may each be in fluid communication with chamber 108A/108B. In alternative embodiments, the bladder may be separated from the chamber 108A/108B by a pressure membrane capable of transmitting force from the bladders 110A/110B to the respective chambers 108A/108B. First bladder 110A/110B and second bladder 112A/112B may each contain a fluid material therewithin. The fluid material may comprise a silicone based gel and/or other, similar fluid materials suitable for such optic applications as will be understood in the art. First haptic element 104A/104B may be connected to first bladder 110A/110B. Second haptic element 106A/106B may be connected to second bladder 112A/112B. In the embodiment, illustrated in FIG. 2A for example, first bladder 110A defines a junction between first haptic element 104A and chamber 108A. Similarly, second bladder 112A defines a junction between second haptic element 106A and chamber 108A. In an alternative embodiment, illustrated in FIGS. 2 and 3A for example, first bladder 110B may be positioned between first haptic element 104B and peripheral edge 120B of lens element 102B. Second bladder 112B may be positioned between second haptic element 106B and peripheral edge 122B of lens element 102B.

A first intermediate membrane defining first bladder 110A/110B may be attached to first haptic element 104A/104B and lens element 102A/102B and a second intermediate membrane defining second bladder 112A/112B may be attached to second haptic element 106A/106B and lens element 102A/102B. The first and second intermediate membranes may be attached via various bonding techniques, such as via a plasma or adhesive bonding. The first and second intermediate membranes may form sacs (as the bladders) that contain the fluid within. The sacs may be of different shapes as illustrated in FIGS. 2A and 3A. The membranes/sacs may be formed with or as a part of the lens body.

Lens element 102A/102B, first haptic element 104A/104B and/or second haptic element 106A/106B generally will be formed of soft, flexible and typically hydrophilic materials, such as silicone, acrylics (for example, AcrySof®), hydrogels and/or combinations thereof. Materials used to form first bladder 110A/110B and second bladder 112A/112B (i.e., the intermediate membranes defining the first bladder 110A/110B and second bladder 112A/112B) can be the same as those of the lens element and haptic elements, for example, where the bladders are integrally formed with the body of the lens element, or may be different from those used to form lens element 102A/102B, first haptic element 104A/104B and/or second haptic element 106A/106B. Also, the fluid material contained within the first bladder 110A/110B and second bladder 112A/112B may be different from the material used to form the first bladder 110A/110B and second bladder 112A/112B. The material used to form the first bladder 110A/110B and second bladder 112A/112B may be impermeable to the fluid contained therein.

First bladder 110A/110B comprises a first compressible body (formed by the first intermediate membrane) mounted along peripheral edge 120A/120B of lens element 102A/102B. Similarly, second bladder 112A/112B comprises a second compressible body (formed by the second intermediate membrane) mounted along peripheral edge 122A/122B of lens element 102A/102B. In particular, “compressible” in this context refers to the bladder yielding to the relatively stiff haptics without deforming the haptics. As further indicated in FIGS. 2B and 4B, first bladder 110A/110B has a first orifice defined therein and through peripheral edge 120A/120B of lens element 102A/102B, extending between first compressible body and chamber 108A/108B of lens element 102A/102B for passage of fluid therebetween. Similarly, second bladder 112A/112B has a second orifice defined therein and through peripheral edge 122A/122B of lens element 102A/102B, extending between second compressible body and chamber 108A/108B for passage of fluid therebetween. The walls of first bladder 110A/110B and second bladder 112A/112B are of sufficient strength such that under compression, they do not bulge out. Instead, the fluid within the first bladder 110A/110B and second bladder 112A/112B is forced through the respective orifices into chamber 108A/108B. Also, thickness of optic membrane 310A maybe greater near the periphery and thinner at the center which causes the center of optic membrane 310A to bulge when fluid is forced into chamber 108A/108B.

Movement of first haptic element 104A/104B causes fluid contained within first bladder 110A/110B to move between first bladder 110A/110B and chamber 108A/108B. Movement of second haptic element 106A/106B causes fluid contained within second bladder 112A/112B to move between second bladder 112A/112B and chamber 108A/108B. Movement of first haptic element 104A/104B and second haptic element 106A/106B is caused by contraction or expansion of a ciliary body of the wearer's eye in which IOL 100A/100B is placed. First haptic element 104A/104B and second haptic element 106A/106B are moveable toward peripheral edge 120A/120B and 122A/122B, respectively, of lens element 102A/102B by contraction of the ciliary body (i.e., when the eye undergoes accommodation). First haptic element 104A/104B and second haptic element 106A/106B are moveable away from peripheral edge 120A/120B and 122A/122B, respectively, of lens element 102A/102B by expansion of the ciliary body (i.e., when the eye undergoes disaccommodation and the ciliary muscle relaxes).

Prior to accommodation or when the eye is in a disaccommodated state, IOL 100A/100B floats in the capsular bag (not otherwise illustrated in the figures) and is held by zonules. In this state, first haptic element 104A/104B and second haptic element 106A/106B barely contact the ciliary body (i.e., are not affixed to the ciliary body).

When the eye undergoes accommodation, the ciliary body contracts. Contraction of the ciliary body causes engagement of the ciliary body with the first haptic element 104A/104B and second haptic element 106A/106B. Such engagement causes movement of first haptic element 104A/104B toward peripheral edge 120A/120B of lens element 102A/102B and movement of second haptic elements 106A/106B toward peripheral edge 122A/122B of lens element 102A/102B. Movement of first haptic element 104A/104B and second haptic element 106A/106B causes compression of first bladder 110A/110B and second bladder 112A/112B, respectively. Compression of first and second bladders 110A/110B, 112A/112B urges fluid from each bladder into chamber 108A/108B of lens element 102A/102B and causes bulging of optic membrane 310A and steepening of the lens radius thereof to adjust focus of lens element 102A/102B for nearer objects. The fluid is urged through orifices into chamber 108A/108B of lens element 102A/102B. The orifices may comprise slots, circular holes, and/or other openings that allow transfer of fluid. Steepening of the lens radius increases the power of lens element 102A/102B which brings nearer objects into focus.

When the ciliary body relaxes (during disaccommodation), the compressive force on first and second haptic elements 104A/104B, 106A/106B is released. In other words, first haptic element 104A/104B and second haptic element 106A/106B move away from the peripheral edge 120A/120B, 122A/122B, respectively, of lens element 102A/102B. Such movement causes decompression of first and second bladders 110A/110B, 112A/112B. Decompression of first and second bladders 110A/110B, 112A/112B enables fluid to move from chamber 108A/108B to each bladder causing flattening of the lens radius of optic membrane 310A to adjust focus of lens element 102A/102B for distance vision. Fluid may be transferred back from chamber 108A/108B to the bladders 110A/110B, 112A/112B via orifices. Flattening of the lens radius reduces the power of lens element 102A/102B back to its resting state for distance vision.

It will be understood that while IOL 100A/100B is described as having two haptic elements, any number of haptic elements may be used to support lens element 102A/102B as long as lens element 102A/102B is centered with respect to the haptics, without departing from the scope of this disclosure.

It further will be understood by those skilled in the art that while the present invention has been described above with reference to preferred embodiments, numerous variations, modifications, and additions can be made thereto without departing from the spirit and scope of the present invention as set forth in the appended claims.

What is claimed is:

1. An intraocular lens adapted to be inserted into a wearer's eye for adjusting the vision thereof, comprising:
a lens element comprising a chamber and an optic membrane along the chamber;
at least one bladder in fluid communication with the chamber, the
at least one bladder containing a fluid material therewithin, and
at least one haptic element connected to the at least one bladder; and
wherein movement of the at least haptic element causes movement of the fluid material between the at least one bladder and the chamber of the lens element so as to cause movement of the optic membrane to vary a lens radius of the optic membrane.

2. The intraocular lens of claim 1, wherein the at least one bladder is positioned between the at least one haptic element and a peripheral edge of the lens element.

3. The intraocular lens of claim 1, wherein the at least one bladder defines a junction between the at least one haptic element and the chamber of the lens element.

4. The intraocular lens of claim 1, wherein the at least one haptic element is moveable toward and away from a peripheral edge of the lens element by contraction and expansion of a ciliary body of the wearer's eye in which the intraocular lens is placed.

5. The intraocular lens of claim 4, wherein movement of the at least one haptic element away from the peripheral edge causes decompression of the at least one bladder, enabling the fluid material to move from the chamber of the lens element to the at least one bladder causing flattening of the lens radius of the optic membrane to adjust focus of the lens element for distance vision.

6. The intraocular lens of claim 4, wherein movement of the at least one haptic element toward the peripheral edge of the lens element causes compression of the at least one bladder urging the fluid material into the chamber to cause bulging of the optic membrane and steepening of the lens radius thereof to adjust focus of the lens element for nearer objects.

7. The intraocular lens of claim 1, wherein at least one bladder comprises a compressible body mounted along a peripheral edge of the lens element and having an orifice defined therein and through the peripheral edge of the lens element, extending between the compressible body and the chamber of the lens element for passage of the fluid material therebetween.

8. The intraocular lens of claim 1, wherein the at least one haptic element contacts the at least one bladder at an angle.

9. The intraocular lens of claim 1, wherein the at least one haptic element comprises a pair of haptic elements connected to the lens element on opposite sides thereof, and the at least one bladder comprises a pair of bladders, each positioned between one of the haptic elements and a peripheral edge of the lens element, and each in fluid communication with the chamber of the lens element.

10. An intraocular lens adapted to be inserted into a wearer's eye for adjusting the vision thereof, comprising:
a lens element comprising a lens body defining a chamber containing a fluid material and an optic membrane along the chamber;
at least one haptic element;
at least one bladder positioned between the at least one haptic element and a peripheral edge of the lens element, the at least one bladder containing a fluid material therewithin; and
wherein movement of the at least haptic element causes movement of the fluid material within the at least one bladder and the chamber of the lens element so as to cause movement of the optic membrane to vary a lens radius of the optic membrane.

11. The intraocular lens of claim 10, wherein the at least one haptic element is moveable toward and away from a peripheral edge of the lens element by contraction and expansion of a ciliary body of the wearer's eye in which the intraocular lens is placed.

12. The intraocular lens of claim 11, wherein movement of the at least one haptic element away from the peripheral edge causes decompression of the at least one bladder to release pressure on the fluid material within the bladder and the chamber thereby causing flattening of the lens radius of the optic membrane to adjust focus of the lens element for distance vision.

13. The intraocular lens of claim 11, wherein movement of the at least one haptic element toward the peripheral edge of the lens element causes compression of the at least one bladder forcing the fluid material within the bladder against sides of the lens body to cause the fluid material within the chamber to move upward thereby causing bulging of the optic membrane and steepening of the lens radius thereof to adjust focus of the lens element for nearer objects.

14. The intraocular lens of claim 10, wherein at least one bladder comprises a compressible body mounted along a peripheral edge of the lens element and having an orifice defined therein and through the peripheral edge of the lens element, extending between the compressible body and the chamber of the lens element for passage of the fluid material therebetween.

15. The intraocular lens of claim 10, wherein the at least one haptic element comprises a pair of haptic elements connected to the lens element on opposite sides thereof, and the at least one bladder comprises a pair of bladders, each positioned between one of the haptic elements and a peripheral edge of the lens element.
The intraocular lens of claim 11, further comprising a membrane between the at least one bladder and the chamber, wherein movement of the at least one haptic element toward the peripheral edge of the lens element causes compression of the at least one bladder forcing the fluid material within the bladder against the membrane to cause the fluid material within the chamber to move upward thereby causing bulging of the optic membrane.
